(19) United States
(12) Patent Application Publication
(10) Pub. No.: US 2025/0042863 A1
(43) Pub. Date: Feb. 6, 2025

(54) METHOD FOR PRODUCING ISOXAZOLINECARBOXYLIC ACID DERIVATIVES

(71) Applicant: BAYER AKTIENGESELLSCHAFT, Leverkusen (DE)

(72) Inventors: Denise CIBU, Leverkusen (DE); Diana Isabel YEPES TEJADA, Leverkusen (DE); Anton LISHCHYNSKYI, Leverkusen (DE); Michael Edmund BECK, Leverkusen (DE); Mark James FORD, Leverkusen (DE); Frank MEMMEL, Leverkusen (DE); Andreas REMBIAK, Leverkusen (DE); Detlev SÜLZLE, Leverkusen (DE)

(21) Appl. No.: 18/716,645

(22) PCT Filed: Dec. 1, 2022
(86) PCT No.: PCT/EP2022/083991

§ 371 Date: Jun. 5, 2024

(30) Foreign Application Priority Data
Dec. 7, 2021 (EP) 21212862.3

(51) Int. Cl. C07D 261/04

(52) U.S. Cl. CPC C07D 261/04

(57) ABSTRACT

The present invention relates to a novel process for preparing isoxazolinecarboxylic acid derivatives of the formula (I).

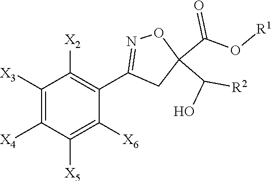

The present invention relates to a novel process for preparing isoxazolinecarboxylic acid derivatives of the formula (I).

Isoxazolinecarboxylic acid derivatives of the general formula (I) are important precursors for active agrochemical ingredients (for example for herbicides that are described in WO2014/048882 and WO2018/228985).

The prior art describes numerous cyclization methods for preparation of isoxazolinecarboxylic acid derivatives, for example Tetrahedron Letters, 1991, 6367-6370; Eur. J. Org. Chem. 2008, 5446-5460; Bull. Chem. Soc. Jpn. 1993, 2685. Possible transition states for the cycloaddition are discussed. Also disclosed are yields and isomer ratios depending on the reaction conditions.

If the compounds of the present invention are obtained by one of the methods known from the literature, this results in yields and isomer purities that are inadequate for an industrial scale synthesis.

It was thus an object of the invention to provide a process for preparing isoxazolinecarboxylic acid derivatives of the general formula (I), which is suitable for synthesis on an industrial scale and has a high yield and isomer purity, such that laborious purification methods can be dispensed with.

The object was achieved in accordance with the invention by a process for preparing isoxazolinecarboxylic acid derivatives of the general formula (I)

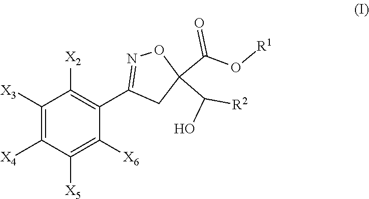

in which
X² is H, C₁-C₄ alkyl, C₁-C₄ fluoroalkyl, C₁-C₄ fluoroalkoxy, C₁-C₄ alkoxy, fluorine, CN,
X³ is H, C₁-C₄ alkyl, C₁-C₄ fluoroalkyl, C₁-C₄ fluoroalkoxy, C₁-C₄ alkoxy, fluorine, chlorine, CN,
X⁴ is H, C₁-C₄ alkyl, C₁-C₄ fluoroalkyl, C₁-C₄ fluoroalkoxy, C₁-C₄ alkoxy, fluorine, CN,
X⁵ is H, C₁-C₄ alkyl, C₁-C₄ fluoroalkyl, C₁-C₄ fluoroalkoxy, C₁-C₄ alkoxy, fluorine, chlorine, CN,
X⁶ is H, C₁-C₄ alkyl, C₁-C₄ fluoroalkyl, C₁-C₄ fluoroalkoxy, C₁-C₄ alkoxy, fluorine, CN,
R¹ is H, C₁-C₁₂-alkyl, unsubstituted benzyl or mono- or di-C₁-C₄-alkyl-substituted benzyl,
R² is C₁-C₄-alkyl,
characterized in that the compounds of the general formula

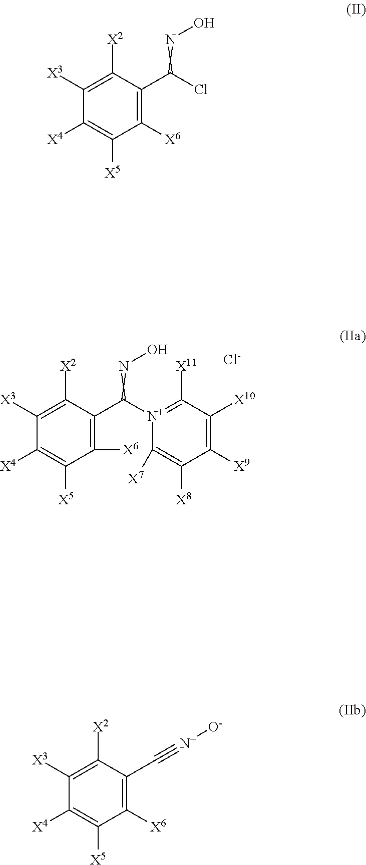

in which
X² to X⁶ have the definitions given above,
X⁷, X⁸, X¹⁰, X¹¹ are independently H or C₁-C₄-alkyl,
X⁹ are H, C₁-C₄-alkyl or N(C₁-C₄-alkyl)₂,
are reacted with compounds of the formula (III)

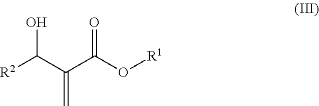

in which
R¹ and R² have the definitions given above,
with addition of an additional base and of a combination of reagents that enables formation of 1 to 3.5 equivalents—based on compounds of the general formula (II)—of a reactive species “R³OMgHal” (IV), where
R³ is alkyl, unsubstituted or alkyl-substituted benzyl and
Hal is halogen,
to give compounds of the general formula (I).

Preferred definitions of the radicals in the compounds of the general formulae (I), (II) and (III) are as follows:
X² is H, methyl, trifluoromethyl, difluoromethyl, difluoromethoxy, trifluoromethoxy, fluorine, methoxy, CN,
X³ is H, methyl, trifluoromethyl, difluoromethyl, difluoromethoxy, trifluoromethoxy, fluorine, chlorine, methoxy, CN,
X⁴ is H, methyl, trifluoromethyl, difluoromethyl, difluoromethoxy, trifluoromethoxy, fluorine, methoxy, CN,
X⁵ is H, methyl, trifluoromethyl, difluoromethyl, difluoromethoxy, trifluoromethoxy, fluorine, chlorine, methoxy, CN,
X⁶ is H, methyl, trifluoromethyl, difluoromethyl, difluoromethoxy, trifluoromethoxy, fluorine, methoxy, CN,
X⁷, X⁸, X¹⁰, X^{u} are independently H, methyl, ethyl,
X⁹ is H, methyl, ethyl or N(methyl)₂
R¹ is H, C₁-C₄-alkyl,
R² is methyl, ethyl.

Preferably, the compounds of the general formula (IV) are generated by one of the following combinations of reagents:
R⁴MgHal and R³OH or
MgHal₂ and R³OM or
MgHal₂ and Mg(OR³)₂, where
R³ is C₂-C₁₂-alkyl, unsubstituted or C₁-C₄-alkyl-substituted benzyl and
Hal is halogen,
M is alkali metal,
R⁴ is alkyl, unsubstituted aryl, substituted aryl, unsubstituted benzyl, substituted benzyl, allyl, vinyl.

Alternatively, the compounds of the general formula (IV) are generated by the following combination of reagents:
R⁵MgHal and R⁶R⁷CO where
R⁵ is C₁-C₆-alkyl, aryl, benzyl,
R⁶, R⁷ are H, C₁-C₆-alkyl, aryl, and the resulting radical definition
R³ corresponds to R⁵R⁶R⁷C.

Preferred radical definitions for the reagents for preparation of compounds of the general formula (IV), presented by way of alternative, are as follows:
R⁵ is C₁-C₄-alkyl, phenyl, benzyl, p-tolyl,
R⁶, R⁷ is H, C₁-C₄-alkyl, phenyl.

Particularly preferred definitions of the radicals in the compounds of the general formulae (I), (II) and (III) are as follows:
X² is H,
X³ is H, methyl, trifluoromethyl, difluoromethyl, fluorine, chlorine, methoxy, CN,
X⁴ is fluorine, H,
X⁵ is H, methyl, trifluoromethyl, difluoromethyl, fluorine, chlorine, methoxy, CN,
X⁶ is H,
X⁷, X⁸, X¹¹ are independently H, methyl, ethyl,
X⁹ is H, N(methyl)₂
X¹⁰ is H,
R¹ is H, methyl, ethyl, i-propyl, i-butyl,
R² is methyl.

More preferably, the compounds of the general formula (IV) are generated by one of the following combinations of reagents:
R⁴MgHal and R³OH or
MgHal₂ and R³OM or
MgHal₂ and Mg(OR³)₂, where
R³ is C₂-C₈-alkyl,
Hal is bromine, chlorine,
M is alkali metal,
R⁴ is C₁-C₈-alkyl, phenyl, benzyl, p-tolyl, vinyl.

Very particularly preferred definitions of the radicals in the compounds of the general formulae (I), (II) and (III) are as follows:
X² is H,
X³ is H, fluorine,
X⁴ is H, fluorine,
X⁵ is H, fluorine,
X⁶ is H,
X⁸ is H, methyl, ethyl,
X⁷, X¹¹ is independently H, methyl,
X⁹, X¹⁰ is H,
R¹ is H, methyl, i-propyl, i-butyl,
R² is methyl.

Even more preferably, the compounds of the general formula (IV) are generated by one of the following combinations of reagents:
R⁴MgHal and R³OH or
MgHal₂ and R³OM or
MgHal₂ and Mg(OR³)₂, where
R³ is i-propyl, i-butyl, 2-butyl,
Hal is bromine, chlorine,
M is sodium,
R⁴ is methyl, ethyl, n-butyl, i-propyl.

Most preferred definitions of the radicals in the compounds of the general formulae (I), (II) and (III) are as follows:
X² is H,
X³ is fluorine,
X⁴ is H,
X⁵ is fluorine,
X⁶ is H,
X⁷, X⁸, X¹¹ is independently H, methyl,
X⁹, X¹⁰ is H,
R¹ is H, methyl, i-butyl,
R² is methyl.

### Definitions

Alkyl means saturated, straight-chain or branched hydrocarbyl radicals having the number of carbon atoms specified in each case, e.g. C₁-C₁₂-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Hal means halogen and is fluorine, chlorine, bromine or iodine. If the term is used for a radical, Hal means a fluorine, chlorine, bromine or iodine atom.

Alkali metal means lithium, sodium or potassium.

Aryl means phenyl or naphthyl.

The compounds of the formula (I) may take the form of isomer mixtures. The desired diastereomeric excess is increased by the optimized reaction conditions compared to the prior art. 1.8 to 2.2 equivalents of a reactive species “R³OMgHal” (IV) is especially advantageous.

When an (S)-alcohol is used, the main product is the (S,S)-diastereomer, and the (R,S)-diastereomer is the secondary diastereomer. When an (R)-alcohol is used, the main product is the (R,R)-diastereomer, and the (S,R)-diastereomer is the secondary diastereomer. When a (rac)-alcohol is used, the main product is the (S,S/R,R)-rac-diastereomer and the (S,R/R,S)-rac-diastereomer is the secondary diastereomer.

The compounds of the formula (I) may be isolated as the corresponding ester or as the carboxylic acid after a hydrolytic workup.

The diastereomer ratio of up to 100:0 may be achieved in the compounds of the formula (I) via an enrichment by crystallization.

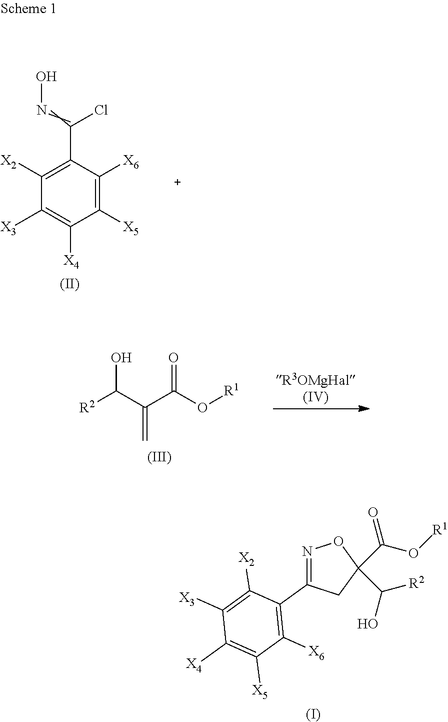

The object was achieved by a process for preparing isoxazolinecarboxylic acid derivatives of the formula (I), characterized in that the compounds of the general formula (II) are reacted with compounds of the formula (III) with addition of an additional base and of a combination of reagents that enables formation of 1 to 3.5 equivalents—based on compounds of the general formula (II)—of a reactive species “R³OMgHal” (IV) to give compounds of the general formula (I) (Scheme 1).

Preference is given to the use of 1.5 to 3.0 equivalents of the reactive species “R³OMgHal” (IV) (based on compounds of the general formula (II)).

Preference is also given to the use of 0.05 to 3.0 equivalents of an additional base (based on compounds of the general formula (II)).

Particular preference is given to the use of 0.1 to 1.5 equivalents of an additional base (based on compounds of the general formula (II)).

Very particular preference is given to the use of 0.2 to 1.0 equivalents of an additional base (based on compounds of the general formula (II)).

The additional base is any base; preference is given to tertiary amines, pyridines and amide bases. Particularly preferred bases are tertiary amines with N(C₁-C₄-alkyl)₃— where two or three different alkyl substituents preferably occur: triethylamine [Et₃N], tributylamine, dii sopropylethylamine, cyclohexyldimethylamine [CyN(methyl)₂], 2- and 3-picoline, methylpyridine, methylethylpyridine, ethylpyridine. Very particular preference is given to Et₃N and CyN(methyl)₂.

Other particular preferred bases are amide bases, for example N,N-dimethylformamide (DMF), N,N-diethylformamide (DEF), dimethylacetamide (DMAc), N,N-dibutylformamide (DBF) or N-methylpyrrolidone (NMP) Also particularly preferred are combinations of a tertiary amine base or of a pyridine base with an amide base.

Very particular preference is given to a combination of a tertiary amine base with an amide base.

The addition of water may also be advantageous and may lead to a further improvement in the diastereomer ratio (d.r.). Preference is given to the use of up to 1.0 equivalent of water (based on compounds of the general formula (II)).

The cyclization is usually conducted within a temperature range from −25° C. to 70° C., preferably 10° C. to 30° C.

In addition, the cyclization is optionally conducted in the presence of a solvent or diluent or of a solvent mixture. The solvents are preferably toluene, xylene, tetrahydrofuran (THF), isopropyl acetate (i-PrOAc), acetonitrile, methyl tert-butyl ether (MTBE), methyl-THF, ethyl acetate (EtOAc) or mixtures in any ratios thereof.

The compounds of the general formula (III) are prepared via a two-stage process, and are known from the literature. The first stage is a Baylis-Hillman reaction. A relevant literature reference is: Drewes, S. E.; Hoole, R. F. A. [Synthetic Communications, 1985, vol. 15, 12, p. 1067-1074]. For the second stage, a relevant literature reference is: Nascimento et al. (2003, Tetrahedron Asymmetry 14, 311-311).

The compounds of the general formulae (II) and (III) are also known from WO 2018/228985. The preparation of the compounds of the formula (ha) from (II) is known from Binenfeld, Zlatko; et al Glasnik Hemijskog Drustva Beograd (1966), 31(4-6), 243-50 and Daroszewski, J.; et al Pharmazie (1986), 41(10), 699-702.

Table 1 shows various possible combinations of reagents, although this selection is not exhaustive.

### EXAMPLES

The present invention is elucidated in detail by the examples that follow, without restricting the invention thereto.

### Analysis Methods

The products were characterized by ¹H NMR spectroscopy and/or LC-MS (Liquid Chromatography Mass Spectrometry).

The NMR spectra were determined using a Bruker Avance 400 fitted with a flow probe head (volume 60 μl). In individual cases, the NMR spectra were measured with a Bruker Avance II 600. In quantitative NMR (gNMR) measurements, methyl 3,5-dinitrobenzoate was used as internal standard.

| | | | | | Additional | | |
|---|---|---|---|---|---|---|---|
| | | i-PrOMgCl, | Additional | Additional | water/ | qNMR | |
| Example | Reaction | equivalents | amine | amide | water in | yield, | Diastereomer |
| No. | procedure | (eq.) | base, eq. | base, eq. | (II), eq. | % | ratio, d.r. |
| 1 | A | 2 | — | — | <0.05 | 80 | 88:12 |
| 2 | A | 2 | — | DBF, | <0.05 | 81 | 92:8 |
| | | | | 1.5 | | | |
| 3 | A | 2 | NEt₃, 1 | DBF, | <0.05 | 83 | 93:7 |
| | | | | 1.5 | | | |
| 4 | A | 3 | — | DBF, | <0.05 | 85 | 92.5:7.5 |
| | | | | 1.5 | | | |
| 5 | A | 3 | — | — | <0.05 | 82 | 90:10 |
| 6 | B | 2 | — | DMF, | <0.05 | 83 | 91:9 |
| | | | | 0.3 | | | |
| 7 | A | 2 | NEt₃, 1 | — | <0.05 | 83 | 90:10 |
| 8 | A | 2 | NEt₃, 1 | DMF, | <0.05 | 81 | 93:7 |
| | | | | 0.5 | | | |
| 9 | B | 2 | NEt₃, 1 | DMF, | <0.05 | 82 | 93.5:6.5 |
| | | | | 0.3 | | | |
| 10 | B | 2* | — | DMF, | 0.07 | 87 | 92.5:7.5 |
| | | | | 0.3 | | | |
| 11 | B | 2 | — | DMF, | 0.3 | 84 | 92:8 |
| | | | | 0.3 | | | |
| 12 | A | 2 | NEt₃, 1 | — | 0.3 | 84 | 92:8 |
| 13 | A | 2 | NEt₃, 1 | DMF, | 0.3 | 80 | 88:12 |
| | | | | 0.3 | | | |
| 14 | A | 2 | NEt₃, 1 | DMAc, | 0.3 | 81 | 92:8 |
| | | | | 0.3 | | | |
| 15 | B | 2 | Me2NCy, 1 | DMF, | 0.3 | 83 | 93:7 |
| | | | | 0.3 | | | |
| 16 | A | 2 | i-Pr₂NEt, 1 | DMF, | 0.3 | 87 | 93:7 |
| | | | | 0.3 | | | |
| 17 | C | 2** | NEt₃, 1 | — | 0.3 | 81 | 91:9 |
| 18 | C | 2** | — | DMF, | 0.3 | 81 | 91:9 |
| | | | | 0.3 | | | |
| 19 | C | 2** | NEt₃, 1 | DMF, | 0.3 | 84 | 93:7 |
| | | | | 0.3 | | | |

*t-BuOMgCl was used
**i-PrOMgCl prepared from MeMgCl and CH₃CHO in THF at 0° C.

Conditions from the prior art applied to the inventive reaction (Example 20)

| | | | | | |
|---|---|---|---|---|---|
| Carreira | Cyclic | 3.3 eq i-PrOH, | CH₂Cl₂, 0° C.-RT | 70% | 86:14 |
| Angew. Chem. | and | 3 eq EtMgBr | | | |
| Int. Ed. 2001, | acyclic | | | | |
| 40, 2082; J. | allyl | | | | |
| Am. Chem. | alcohols | | | | |
| Soc. 2001, 123, | | | | | |
| 3611; Org. | | | | | |
| Lett. 2007, 9, | | | | | |
| 3857 | | | | | |

Prior art (X₂₋₆ = H)

| | | | | | |
|---|---|---|---|---|---|
| Bull. Chem. | Methyl, | 2 eq EtMgBr | CH₂Cl₂, −78 to −30° C./ | 100 | 92:8/89:11 |
| Soc. Jpn. | 2 eq | | to R.T. | | |
| 1993, 2685- | Methyl, | 1 eq NEt₃ | CH₂Cl₂, −30° C. | 90 | 30:70 |
| 2689 | 1 eq | | | | |
| | Methyl, | 1 eq EtMgBr | THF/CH₂Cl₂, −78 to | 93/86 | 63:37/81:19 |
| | 1 eq | | −30° C. | | |

### Example 3 (Reaction Procedure Type A)

Under an argon atmosphere, 2 equivalents (eq) of 2-propylmagnesium chloride (4.00 ml, 2 mol/1 in THF) is initially charged at 20° C. 2 eq of 2-propanol (0.61 ml) is added dropwise over the course of 10 min while cooling (ice bath). Propane escapes (foaming only slightly), and a white solid precipitates out. After the addition has ended and once the evolution of gas has ended, the resultant suspension is stirred at 20° C. for 20 minutes. Subsequently, 1 eq of methyl 3-hydroxy-2-methylenebutanoate (0.49 ml, 99.8% by weight) is added at RT and stirring is continued for 15 min. The mixture becomes more fluid. Thereafter, 1 eq of triethylamine (0.56 ml) is added dropwise at RT over the course of 5 min while stirring. After stirring at RT for a further 15 min, the solution of 3,5-difluoro-N-hydroxybenzenecarboximidoyl chloride in a solvent mixture of toluene/THF (4.14 g, 18.5% by weight) is added dropwise by means of a syringe pump together with an additional 1.5 eq of N,N-di-N-butylformamide (DBF) (1.10 ml) at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. An excess of aqueous sodium hydroxide solution is added to the residue, and it is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (qNMR), 0.90 g of product is present in the residue (83%). Diastereomer ratio=93:7 (HPLC).

### Example 15 (Reaction Procedure Type B)

Under an argon atmosphere, 2 equivalents (eq) of 2-propylmagnesium chloride (4.00 ml, 2 mol/l in THF) is initially charged at 20° C. 2 eq of 2-propanol (0.61 ml) is added dropwise over the course of 10 min while cooling (ice bath). Propane escapes (foaming only slightly), and a white solid precipitates out. After the addition has ended and once the evolution of gas has ended, the resultant suspension is stirred at 20° C. for 20 minutes. Subsequently, 1 eq of methyl 3-hydroxy-2-methylenebutanoate (0.49 ml, 99.8% by weight) is added at RT and stirring is continued for 15 min. The mixture becomes more fluid. Thereafter, 1 eq of N,N-dimethylcyclohexylamine (0.60 ml) is added dropwise at RT over the course of 5 min while stirring. After stirring at RT for a further 15 min, 0.3 eq of DMF (92 μl) is added, and the suspension is stirred at RT for a further 10 min and then cooled to about 15° C. Subsequently, the solution of 3,5-difluoro-N-hydroxybenzenecarboximidoyl chloride in a solvent mixture of toluene/THF (3.33 g, 23.00% by weight) is added dropwise by means of a syringe pump together with an additional 0.3 eq of water (32 μl) at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. An excess of aqueous sodium hydroxide solution is added to the residue, and it is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (gNMR), 0.89 g of product is present in the residue (82%). Diastereomer ratio=94:6 (HPLC).

### Example 19 (Reaction Procedure Type C)

Under an argon atmosphere, 2 equivalents (eq) of methylmagnesium chloride (2.67 ml, 3 mol/1 in THF) is initially charged at 0° C. (ice bath cooling). A solution of 2.075 eq of acetaldehyde in dry THE (0.47 ml in 1.25 ml of THF) is added dropwise at 0° C. while stirring over the course of 10 minutes (min). A white solid precipitates out during the addition. The suspension is stirred at 0° C. for a further 30 min and then warmed to room temperature (RT) over the course of about 15 min. Subsequently, 1 eq of methyl (3S)-3-hydroxy-2-methylenebutanoate (0.52 ml, 95.5% by weight, 98.8% e.e.) is added at RT and stirring is continued for 15 min. The mixture becomes more fluid. Thereafter, 1 eq of triethylamine (0.56 ml) is added dropwise at RT over the course of 5 min while stirring. After stirring at RT for a further 15 min, 0.3 eq of DMF (92 μl) is added, and the suspension is stirred at RT for a further 10 min and then cooled to about 15° C. Subsequently, the solution of 3,5-difluoro-N-hydroxybenzenecarboximidoyl chloride in a solvent mixture of toluene/THF (2.58 g, 29.6% by weight) is added dropwise by means of a syringe pump together with an additional 0.3 eq of water (22 μl) at about 15° C. The addition time is 1 hour (h). Thereafter, the reaction mixture is warmed to RT and stirred for a further 1 h. Then HCl solution is added to the reaction mixture, and it is extracted with ethyl acetate. The water phase is extracted once again with ethyl acetate. The combined organic phases are concentrated under reduced pressure. An excess of aqueous sodium hydroxide solution is added to the residue, and it is stirred at 65° C. On completion of hydrolysis, the reaction mixture is cooled down to RT, acidified (pH 1-2) and extracted twice with ethyl acetate. The combined organic phases are concentrated under reduced pressure. After determination of the content (gNMR), 0,92 g of product is present in the residue (84%). Diastereomer ratio=93:7 (HPLC).

### Example 20

6.79 g (51.2 mmol) of methyl (3S)-3-hydroxy-2-methylenebutanoate was dissolved in 650 ml of dichloromethane under an argon atmosphere at room temperature, and isopropanol was added. The clear solution was cooled to 0° C. Subsequently, 156 ml (156 mmol) of EtMgBr (1M) in THE was slowly added dropwise (exothermic). The solution turned cloudy. Then a solution of 10.00 g (52.2 mmol) of 3,5-difluoro-N-hydroxybenzimidoyl chloride in 100 ml of DCM was slowly added dropwise while stirring (15 min), and the mixture was warmed gradually to room temperature. The mixture turned a distinct yellow colour. TLC in EA/n-heptane 1:1 showed complete conversion after 30 min.

### Workup

The solution was added to 11 of a 1:1 mixture of 2N HCl and saturated NaCl solution, and extracted twice with 400 ml each time of methylene chloride, dried over Na₂SO₄, filtered and concentrated. The LCMS analysis of the crude product showed a diastereomeric ratio of the products of 86% to 14%.

Chromatography on Silica Gel (n-Hep/EA)

Fr. 1 m=600 mg (4.0%) of lipophilic diastereomer

¹H NMR (CDCl₃): 1.10 (d, 3H, CHCH₃), 2.3 (s br., 1H, OH), 3.53 (d, 1H, CHH isoxazoline), 3.72 (d, 1H, CHH isoxazoline), 3.84b(s, 3H, OCH₃), 4,34 (q, 1H, CHCH₃), 6.88 (tt, 1H, arom. H), 7.22 (m, 2H, arom. H).

Fr. 2 m=7700 mg (51.7%) polar diastereomer

¹H NMR (CDCl₃): 1.29 (d, 3H, CHCH₃), 2.10 (d, 1H, OH), 3.57 (d, 1H, CHH isoxazoline), 3.69 (d, 1H, CHH isoxazoline), 3.84b(s, 3H, OCH₃), 4,24 (q, 1H, CHCH₃), 6.88 (tt, 1H, arom. H), 7.18 (m, 2H, arom. H).

1. A process for preparing isoxazolinecarboxylic acid derivatives of formula (I) 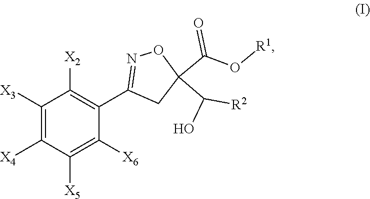
in which
X² is H, C₁-C₄ alkyl, C₁-C₄ fluoroalkyl, C₁-C₄ fluoroalkoxy, C₁-C₄ alkoxy, fluorine, CN,
X³ is H, C₁-C₄ alkyl, C₁-C₄ fluoroalkyl, C₁-C₄ fluoroalkoxy, C₁-C₄ alkoxy, fluorine, chlorine, CN,
X⁴ is H, C₁-C₄ alkyl, C₁-C₄ fluoroalkyl, C₁-C₄ fluoroalkoxy, C₁-C₄ alkoxy, fluorine, CN,
X⁵ is H, C₁-C₄ alkyl, C₁-C₄ fluoroalkyl, C₁-C₄ fluoroalkoxy, C₁-C₄ alkoxy, fluorine, chlorine, CN,
X⁶ is H, C₁-C₄ alkyl, C₁-C₄ fluoroalkyl, C₁-C₄ fluoroalkoxy, C₁-C₄ alkoxy, fluorine, CN,
R¹ is H, C₁-C₁₂-Alkyl, unsubstituted benzyl or mono- or di-C₁-C₄ alkyl-substituted benzyl,
R² is C₁-C₄-alkyl,
wherein at least one compound corresponding to one of general formulae (II), (IIa), and (IIb) 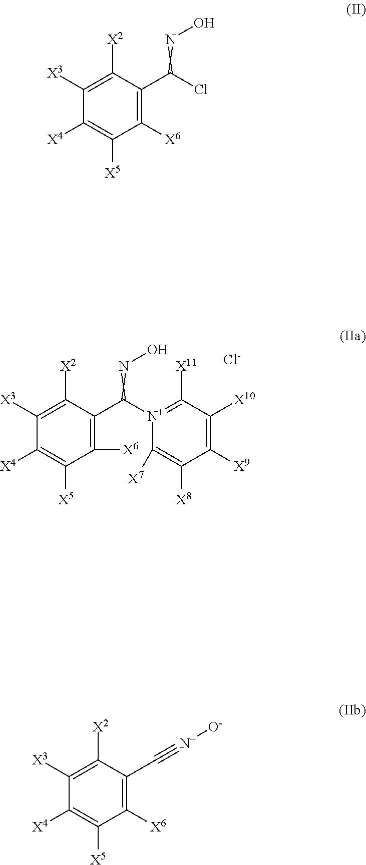
in which
X² to X⁶ have the definitions given above,
X⁷, X⁸, X¹⁰, X¹¹ are independently H or C₁-C₄-alkyl,
X⁹ are H, C₁-C₄-alkyl or N(C₁-C₄-alkyl)₂,
undergo a reaction with compounds of formula (III) 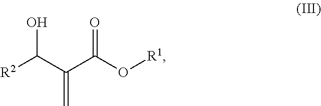
in which
R¹ and R² have the definitions given above,
with addition of an additional base and of a combination of reagents that enables formation of 1 to 3.5 equivalents, based on compounds of the general formulae (II), (IIa), and (IIb), of a reactive species “R³OMgHal” (IV), where
R³ is alkyl, unsubstituted or alkyl-substituted benzyl and
Hal is halogen,
to give compounds of the general formula (I).

2. The process according to claim 1, wherein the definitions of radicals in the compounds of the general formulae (I), (II), (IIa), (IIb, and (III) are as follows:
X² is H, methyl, trifluoromethyl, difluoromethyl, difluoromethoxy, trifluoromethoxy, fluorine, methoxy, CN,
X³ is H, methyl, trifluoromethyl, difluoromethyl, difluoromethoxy, trifluoromethoxy, fluorine, chlorine, methoxy, CN,
X⁴ is H, methyl, trifluoromethyl, difluoromethyl, difluoromethoxy, trifluoromethoxy, fluorine, methoxy, CN,
X⁵ is H, methyl, trifluoromethyl, difluoromethyl, difluoromethoxy, trifluoromethoxy, fluorine, chlorine, methoxy, CN,
X^{°} is H, methyl, trifluoromethyl, difluoromethyl, difluoromethoxy, trifluoromethoxy, fluorine, methoxy, CN,
X⁷, X⁸, X¹⁰, X¹¹ are independently H, methyl, ethyl,
X⁹ is H, methyl, ethyl or N(methyl)₂
R¹ is H, C₁-C₄-alkyl,
R² is methyl, ethyl.

3. The process according to claim 1, wherein the definitions of radicals in the compounds of the general formulae (I), (II), (IIa), (IIb), and (III) are as follows:
X is H,
V is H, methyl, trifluoromethyl, difluoromethyl, fluorine, chlorine, methoxy, CN,
X⁴ is fluorine, H,
X⁵ is H, methyl, trifluoromethyl, difluoromethyl, fluorine, chlorine, methoxy, CN,
X⁶ is H,
X⁷, X⁸, X¹¹ are independently H, methyl, ethyl,
X⁹ is H, N(methyl)₂
X¹⁰ is H,
R¹ is H, methyl, ethyl, i-propyl, i-butyl,
R² is methyl.

4. The process according to claim 1, wherein the definitions of radicals in the compounds of the general formulae (I), (II)(IIa), (IIb) and (III) are as follows:
X² is H,
X³ is H, fluorine,
X⁴ is H, fluorine,
X⁵ is H, fluorine,
X⁶ is H,
X⁸ is H, methyl, ethyl,
X⁷, X¹¹ is independently H, methyl,
X⁹, X¹⁰ is H,
R¹ is H, methyl, i-propyl, i-butyl,
R² is methyl.

5. The process according to claim 1, wherein the definitions of radicals in the compounds of the general formulae (I), (II), (IIa), (IIb), and (III) are as follows:
X² is H,
X³ is fluorine,
X⁴ is H,
X⁵ is fluorine,
X⁶ is H,
X⁷, X⁸, X¹¹ is independently H, methyl,
X⁹, X¹⁰ is H,
R¹ is H, methyl, i-butyl,
R² is methyl.

6. The process according to claim 1, wherein the definitions of radicals in the compounds of the general formula (IIa) are as follows:
X⁷, X⁸, X¹⁰, X¹¹ are independently H, methyl, ethyl,
X⁹ is H, methyl, ethyl, or N(methyl)₂.

7. The process according to claim 6, wherein the definitions of the radicals in the compounds of the general formula (IIa) are as follows:
X⁷, X¹¹ is H,
X⁸, X¹⁰ are independently H, methyl, ethyl,
X⁹ is H, methyl, ethyl, N(methyl)₂.

8. The process according to claim 1, wherein the compound of the general formula (IV) is generated by one of the following combinations of reagents:
R⁴MgHal and R³OH or
MgHal₂ and R³OM or
MgHal₂ and Mg(OR³)₂, where
R³ is C₂-C₁₂-alkyl, unsubstituted or C₁-C₄-alkyl-substituted benzyl and
Hal is halogen,
M is alkali metal,
R⁴ is alkyl, unsubstituted aryl, substituted aryl, unsubstituted benzyl, substituted benzyl, allyl, vinyl.

9. The process according to claim 1, wherein the compound of the general formula (IV) is generated by one of the following combinations of reagents:
R⁴MgHal and R³OH or
MgHal₂ and R³OM or
MgHal₂ and Mg(OR³)₂, where
R³ is C₂-C₈-alkyl,
Hal is bromine, chlorine,
M is alkali metal,
R⁴ is C₁-C₈-alkyl, phenyl, benzyl, p-tolyl, vinyl.

10. The process according to claim 1, wherein the compound of the general formula (IV) is generated by one of the following combinations of reagents:
R⁴MgHal and R³OH or
MgHal₂ and R³OM or
MgHal₂ and Mg(OR³)₂, where
R³ is i-propyl, i-butyl, 2-butyl,
Hal is bromine, chlorine,
M is sodium,
R⁴ is methyl, ethyl, n-butyl, i-propyl.

11. The process according to claim 1, wherein 1.5 to 3.0 equivalents of the reactive species “R³OMgHal” (IV) is used, based on compounds of the general formula (II).

12. The process according to claim 1, wherein 0.05 to 3.0 equivalents of the additional base is used, based on compounds of the general formula (II).

13. The process according to claim 12, wherein 0.1 to 1.5 equivalents of the additional base is used, based on compounds of the general formula (II).

14. The process according to claim 13, wherein 0.2 to 1.0 equivalents of the additional base is used, based on compounds of the general formula (II).

15. The process according to claim 1, wherein the additional base is Et₃N or CyN(methyl)₂.

16. The process according to claim 1, wherein the reaction is carried out in a solvent, wherein the solvent is toluene, xylene, tetrahydrofuran (THF), isopropyl acetate (i-PrOAc), acetonitrile, methyl tert-butyl ether (MTBE), methyl-THF, ethyl acetate (EtOAc) or mixtures in any ratios thereof.

17. The process according to claim 1, wherein the reaction is conducted at −25° C. to 70° C.

18. The process according to claim 17, characterized in that, wherein the reaction is conducted at 10° C. to 30° C.

19. The process according to claim 1, wherein the base used is a combination of a tertiary amine base or of a pyridine base with an amide base.

20. The process according to claim 1, wherein up to 1.0 equivalent of water is used based on compounds of the general formula formulae (II), (IIa), and (IIb).

21. The process according to claim 1, wherein a diastereomer ratio of the compounds of general formula I is increased by a further crystallization step.

22. The process according to claim 1, wherein the compounds of the general formula (IV) are generated via a Grignard reaction, specifically with the following combinations of reagents:
R⁵MgHal and R⁶R⁷CO where
R⁵ is C₁-C₆-alkyl, aryl, benzyl,
R⁶, R⁷ are H, C₁-C₆-alkyl, aryl, and the resulting radical definition
R³ corresponds to R⁵R⁶R⁷C.
